(19) United States
(12) Patent Application Publication
(10) Pub. No.: US 2003/0207844 A1
(43) Pub. Date: Nov. 6, 2003

(54) NOS inhibitors for treatment of wrinkles

(75) Inventors: Ethan Lerner, Newton, MA (US); Seishiro Fujii, Boston, MA (US)

Correspondence Address: FISH & RICHARDSON PC, 225 FRANKLIN ST, BOSTON, MA, 02110, US

(73) Assignee: The General Hospital Corporation, a Massachusetts corporation

(21) Appl. No.: 10/406,306

(22) Filed: Apr. 3, 2003

Related U.S. Application Data
(62) Division of application No. 10/057,247, filed on Jan. 25, 2002.
(60) Provisional application No. 60/264,176, filed on Jan. 25, 2001.

(51) Int. Cl. A61K 7/42; A61K 31/655

(52) U.S. Cl. 514/149; 424/59

(57) ABSTRACT

Methods, compositions, and kits, are provided for the use of nitric oxide synthase inhibitors to prevent and reduce wrinkles.

### RELATED APPLICATIONS

This application claims the benefit of U.S. Provisional Application Serial No. 60/264,176, filed Jan. 25, 2001, the contents of which are incorporated herein by reference in their entirety.

### BACKGROUND

This invention relates to the use of NOS inhibitors to prevent wrinkles.

Nitric oxide (NO) has been implicated to date in a vast array of physiological processes, including vasodilation, neurotransmission, sensory perception, and immune response (reviewed in Qureshi et al. (1996) Arch Dermatol 132:889-893). Under normal conditions, NO produced in low concentration acts as a messenger and cytoprotective (antioxidant) factor, via direct interactions with transition metals and other free radicals (Liaudet et al. (2000) Crit Care Med 28(4 Suppl):N37-52). At abnormally high levels, NO is cytotoxic, relevant to the pathophysiology of inflammation, circulatory shock, and ischemia-reperfusion injury. Id. Therapeutics that modulate NO levels in human tissue have become a state-of-the-art strategy for targeting cardiovascular and anti-inflammatory indications and sexual dysfunction (Janero (2000) Free Radic Biol Med 28:1495-506).

NO is synthesized by nitric oxide synthase (NOS), which oxidizes the guanidine-nitrogen in L-arginine, producing NO and citrulline. Three NOS isoforms have been characterized: type I, found in neuronal cells; type II, found in macrophages; and type III, found in endothelial cells. NO is actively produced in the skin, where all three of these cell types are found. In the presence of NO, blood flow in the human skin microcirculation is remarkably increased and in the presence of inhibitors of NOS, vasodilatation is impaired (Warren (1994) FASEB J8:247-51; Ralevic et al. (1992) Br J Pharmacol 106:650-655). Other studies of the role of NO and NOS in the skin indicate that NO may be involved in psoriasis, atopic dermatitis, and in the paracrine mediation of UV-induced melanogenesis (Kolb-Bachofen et al. (1994) Lancet 344:139; Morita et al. (1995) Int JDermatol 34:294; Romero-Graillet et al. (1997) J Clin Invest 99:635-642). Additionally, there is evidence that nitric oxide synthesis in a variety of skin cells is modulated by diverse inflammatory and immune stimuli, supporting a role for NO and NOS in the maintenance and physiology of human skin (reviewed in Bruch-Gerharz et al. (1998) J Invest Dermatol 110: 1-7).

### SUMMARY OF THE INVENTION

In general, the invention features, a method of treating skin, e.g., preventing or reducing symptoms of aging skin, e.g., wrinkles, e.g., fine wrinkles; drying; or cracking. The method includes administering to a subject, e.g., a human, an effective amount of a NOS inhibitor, e.g., L-NAME, to prevent, or treat, a symptom of aging skin, e.g., a wrinkle or a fine wrinkle. In a preferred embodiment, the subject's skin has been exposed to radiation, e.g., UV radiation, e.g., to UVB radiation, e.g., the subject has been exposed to the sun, or the subject shows symptoms of aging skin, e.g., wrinkles. In a preferred embodiment, the NOS inhibitor is administered topically. The NOS inhibitor can be administered to the face, chest, neck, hands, and other regions of the body. The treatment can involve more than one administration, e.g., at least two, three, or four administrations, of the NOS inhibitor. The treatment can also involve daily administration of the NOS inhibitor.

In another embodiment, the method includes: identifying a subject in need of preventing or treating wrinkle formation; administering a NOS inhibitor compound; and evaluating the effect of the administration on wrinkle formation. In a preferred embodiment, the subject's skin has been exposed to UV, e.g., UVB radiation. The identification of a subject in need of preventing or reducing wrinkles can be performed e.g., by the subject, by a health care provider, or by a provider of cosmetics. The NOS inhibitor may be administered, e.g., by the subject, by a health care provider, or by a provider of cosmetics. Likewise, the evaluation of the effect of wrinkle formation may be performed, e.g., by the subject, by a health care provider, or by a provider of cosmetics.

The invention also features compositions containing NOS inhibitors for preventing or treating wrinkles, e.g., fine wrinkles. In a preferred embodiment, the NOS inhibitor is provided in a pharmaceutically acceptable composition. In a preferred embodiment, the composition is sterile. In a preferred embodiment, the weight percent of the NOS inhibitor ranges from 0.01% to 10%. In another preferred embodiment, the weight percent of the NOS inhibitor ranges from 0.05% to 10%. The composition is effective to temporarily reduce the appearance of wrinkles when applied to the skin, e.g., for a period of at least 2 to 100 days, more preferably at least 7 to 90 days, even more preferably 14 to 60 days, or it can be effective to reduce the appearance of wrinkles for a longer term, e.g., at least 3 to 9 months, more preferably 4 to 8 months, or about 6 months. In a preferred embodiment, the composition also has a fragrance, a preservative, or other cosmetic ingredient, e.g., a moisturizer, or sunscreen agent, e.g., octyl methoxycinnamate, aminobenzoic acid, oxybenzone, padimate O, homosalate, or titanium dioxide. The composition can be provided in a cream, lotion, foam, gel, or other cosmetic preparation.

In some embodiments, the NOS inhibitor can be modified, e.g., derivatized or conjugated to another molecule. In preferred embodiments, the NOS inhibitor is modified to make it more suitable for human use, e.g., to make the NOS inhibitor more active, more stable, or more soluble.

In another aspect, the invention features a method of providing wrinkle protection to a subject by supplying a NOS inhibitor composition described herein, e.g., L-NAME, to the subject, preferably with instructions to apply prior to, or after, UV exposure, e.g., UVB, e.g., sunlight exposure.

In another aspect, the invention features a kit for providing wrinkle protection to a subject which includes a composition described herein, e.g., a composition containing a NOS inhibitor, e.g., L-NAME; and instructions for use, e.g., instructions to apply the composition prior to, or after, UV, e.g., UVB exposure, e.g., sunlight exposure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications mentioned herein are incorporated by reference in their entirety. In case of conflict, the present specification, including definitions, will control. In addition, the materials, methods, and examples are illustrative only and not intended to be limiting.

Other features or advantages of the present invention will be apparent from the following detailed description of several embodiments, and also from the appending claims.

### DETAILED DESCRIPTION

The invention relates to methods for preventing or reducing wrinkle formation by administering a NOS inhibitor compound to a subject. A preferred NOS inhibitor is L-NAME. Another aspect of this invention features a composition having a NOS inhibitor as an active ingredient.

Wrinkles are generally a result of the natural aging process of the skin, and of exposure to the sun's ultraviolet rays. A wrinkle is a configuration change in the surface of the skin, without specific structural alterations at the histological level. Generally, wrinkles are classified as described in Kligman et al. (1985) Br J Derm 113:37-42, herein incorporated by reference. Kligman classifies wrinkles into three classes: linear wrinkles, glyphic wrinkles, and crinkles. Linear wrinkles are straight, found generally in the facial skin, and are caused by natural aging or exposure to ultraviolet light. Glyphic wrinkles are shaped as apparent triangles or rectangles of wrinkles, are found on the face, hands, and neck exposed to sunlight, and are aggravated by exposure to ultraviolet light or dermatoheliosis. Crinkles are thin, crinkled wrinkles on flabby skin, found anywhere on the skin, but typically on the backs of hands and around the eyelids.

Herein, linear wrinkles are further subclassified into (a) regular wrinkles and (b) fine wrinkles. Regular wrinkles are long, deep, clear, and are also referred to as crow's feet. Fine wrinkles are thin and shallow. Regular wrinkles have a width of at least about 155 microns (0-32 Hz), preferably about 160 to 250 microns. Fine wrinkles have a width of less than about 154 microns, preferably about 40 to 154 microns (32-126 Hz), as calculated e.g., in a power spectrum obtained through transforming three dimensional shape data into data in a frequency domain by two-dimensional Fourier transformation (using, e.g., the Shiseido Wrinkle Analyzer 3D Pro system, essentially as described in Takasu et al. (1996) J Soc Cosmet Chem Japan 29:394-405; and Japanese Published Patent Application No. 07-113623, published May 02, 1995).

The method herein provided to prevent or treat or reduce wrinkles, especially fine wrinkles, in a subject, includes administering to the subject a composition comprising a NOS inhibitor. The NOS inhibitor L-N^{G} nitro-arginine methyl ester (L-NAME), when applied to the back skin of mice at a concentration of 1% in 70% ethanol, prevented the formation of fine wrinkles caused by UVB exposure (Example 2). Other NOS inhibitors suitable for use in the invention include, but are not limited to, N-monomethyl-L-arginine (L-NMMA); N-G monomethyl-L-arginine (NMA); L-NNA; ARL 17477; L-NIL; aminoguadinine; and ADMA. L-NAME is a preferred NOS inhibitor. Other NOS inhibitors have been described in, e.g., Gapud et al., U.S. Pat. No. 5,981,511; Mjalli et al, U.S. Pat. No. 5,723,451; Hallinan et al., U.S. Pat. No. 6,143,790; Hansen et al., U.S. Pat. No. 6,071,906; Hansen et al., U.S. Pat. No. 6,043,261, all of which are herein incorporated by reference.

An effective amount of the composition of the present invention is defined as the amount of the composition which, upon administration to a subject, prevents the formation of wrinkles, or fine wrinkles, in the subject, or reduces the appearance of wrinkles, or fine wrinkles, in the subject. The effective amount to be administered to a subject is typically based on a variety of factors including age, sex, surface area, weight, and conditions of the skin. Body surface area may be approximately determined from height and weight of the patient. See, e.g., Scientific Tables, Geigy Pharmaceuticals, Ardley, N.Y. 1970, 537. Effective doses will vary, as recognized by those skilled in the art, dependant on route of administration, excipient usage, and the possibility of co-usage with other treatments such as usage of other wrinkle reducing compounds.

As used herein, “preventing or treating a wrinkle” means the application or administration of a therapeutic agent to a subject who has a wrinkle, e.g., a fine wrinkle, or has a predisposition toward wrinkles, or has been exposed to an agent likely to cause wrinkles, e.g., UV radiation, e.g., UVB irradiation, with the purpose to reduce, improve, alleviate, alter, remedy, ameliorate, or affect, the appearance of the wrinkle or the formation of the wrinkle. The compound of the invention can be administered to the subject by the subject himself or herself, or by another person, e.g., a health care provider or a provider of cosmetics in preferred embodiments of the methods described herein, wrinkles, e.g., fine wrinkles, are reduced in the subject by at least 5%, preferably at least 10%, more preferably at least 20%, 25% or more.

The methods and compositions can be used prophylactically or they can be used to prevent further wrinkle formation or reduce the appearance of wrinkles in a subject. The use of the composition for the manufacture of a medicament or cosmetic for preventing or treating wrinkles is also within the scope of this invention.

Administration of NOS Inhibitor Compositions

The pharmaceutical composition for the prevention or reduction of wrinkles may be administered via the parenteral route, including orally, topically, subcutaneously, intraperitoneally, intramuscularly, intranasally, and intravenously. Topical administration is preferred. Repeated administration of the composition, e.g., repeated topical administration, can be used. More than one route of administration can be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the wrinkle reducing composition.

The composition of this invention can also be formulated into dosage forms for other routes of administration utilizing conventional methods. A pharmaceutical composition can be formulated, for example, in dosage forms for oral administration in a capsule, a tablet (each including timed release and sustained release formulations), or a gel seal. Capsules may comprise any standard pharmaceutically acceptable material such as gelatin or cellulose derivatives. Tablets may be formulated in accordance with the conventional procedure by compressing mixtures of NOS inhibitor compounds and a solid carrier, and a lubricant. Examples of solid carriers include starch and sugar bentonite. The wrinkle reducing composition can also be administered in a form of a hard shell tablet or capsule containing, for example, lactose or mannitol as a binder and a conventional filler and a tableting agent.

Topical administration of the wrinkle reducing compounds described herein presents a preferred route of administration amongst the many different routes described above. For topical application, the compositions of the present invention can include a medium compatible with skin. Such topical pharmaceutical compositions can exist in many forms, e.g., in the form of a solution, cream, ointment, gel, lotion, shampoo, or aerosol formulation adapted for application to the skin. The weight percent of the active ingredient in the composition, i.e., the NOS inhibitor compound, useful in preventing or reducing wrinkles ranges from 0.01% to 10% (based on the total weight of the composition) in admixture with a pharmaceutically acceptable carrier. A wide variety of carrier materials can be employed in the wrinkle reducing composition of this invention such as alcohols, aloe vera gel, allantoin, glycerine, vitamin A and E oils, mineral oils, and polyethylene glycols. Other additives, e.g., preservatives, fragrance, sunscreen, or other cosmetic ingredients, can be present in the composition. The topical composition can be applied and removed immediately, or it can be applied and left on the skin surface, e.g., the face, for an extended period of time, e.g., overnight or throughout the day.

Measurement of Wrinkles

The effect of a compound on the formation or appearance of wrinkles can be evaluated qualitatively, e.g., by visual inspection, or quantitatively, e.g., by computer assisted measurements of wrinkle morphology. Preferably, wrinkle morphology is quantitatively analyzed. Examples of quantitative methods for measuring wrinkles include, but are not limited to, the optical cut technique employing a laser beam, as proposed by Hoshino (1992) Pixel 45:121, herein incorporated by reference; or methods which analyze three-dimensional skin replicas, e.g., the Shiseido Wrinkle Analyzer 3D Pro system (Takasu et al. (1996) J Soc Cosmet Chem Japan 29:394-405; Japanese Published Patent Application No. 07-113623, published May 02, 1995 (corresponds to U.S. patent application Ser. No. 08/364,346)). The SILFLO (Flexico Development Ltd.) system or a similar system can be used to take a replica of the skin. Irregularities on the surface of the skin replica, i.e., wrinkles, are analyzed, e.g., with the Shiseido Wrinkle Analyzer 3D Pro or a similar system, to provide three-dimensional shape data from the heights at points on a two-dimensional plane corresponding to the skin. According to the three-dimensional data, the length, width, depth, area, and volume of each wrinkle is calculated. According to the parameters for regular and fine wrinkles described herein, different classes of wrinkles, including the subclasses of regular and fine wrinkles, can thus be individually recognized and scored.

The following specific examples, which describe the wrinkle reducing compositions of this invention and biological testings of such compositions, are to be construed as merely illustrative, and not limitative of the remainder of the disclosure in any way whatsoever.

### EXAMPLES

### Example 1

To examine the potential for irritation from topical application of a NOS inhibitor to the skin, measurements of ear swelling response were performed as follows: The thickness of both ears of each of three female Balb/C mice, 10 weeks of age, was measured with a thickness gage (Mitsutoyo Corp.). Ten microliters of 1% L-NAME (L-NG-nitro-arginine methyl ester) in 70% ethanol in water was applied to each right ear, and 10 microliters of 70% ethanol in water was applied to each left ear as a control. Ear thickness was monitored over two consecutive days following administration of the solutions. The measurements from the three mice were averaged and are provided in Table 1. As shown, there is no statistically significant difference in ear thickness between the control and the L-NAME treated ears, indicating that 1% L-NAME in 70% ethanol did not cause skin inflammation. In addition, no other signs of skin irritation or inflammation, such as redness or flaking, were observed in the L-NAME versus the control treated ears.

TABLE 1

| | DAY 0 | DAY 1 | DAY 2 |
|---|---|---|---|
| | (×0.01 mm) | (×0.01 mm) | (×0.01 mm) |
| | AVE ± SD | AVE ± SD | AVE ± SD |
| Left ears (control) | 26.33 ± 0.58 | 25.33 ± 0.58 | 25.67 ± 0.58 |
| Right ears (1% L-NAME) | 26.33 ± 0.58 | 25.33 ÷ 0.58 | 26.33 ± 0.58 |

### Example 2

To examine the effect of L-NAME on wrinkle formation, wrinkle measurements experiments were performed on the back skin of mice (HR-1 hairless strain, female, 5 weeks of age, n=3) by taking and analyzing skin replicas.

Mice were exposed to UVB irradiation three times a week for ten weeks 22 beginning with 0.3 mW/cm². The total exposure was 4.662J/cm². 100 microliters of 1% L-NAME in 70% ethanol, or 70% ethanol as a control, was applied after each UVB irradiation. Three days after the final UVB irradiation, skin replicas of the dorsal area were taken by means of SILFLO (Flexico Development Ltd.). Replicas were evaluated by the Wrinkle Analyzer 3D Pro developed by Shiseido (essentially as described in Takasu et al. (1996) J Soc Cosmet Chem Japan 29:394-405). Wrinkle volume and width of regular and fine wrinkles was calculated through three-dimensional data. The results are provided in Table 2. As can be seen, the L-NAME treated mice show a reduction in fine wrinkles compared to the untreated control mice, indicating that 1% L-NAME in 70% ethanol can prevent the formation of wrinkles caused by UVB exposure.

TABLE 2

| | WRINKLE VOLUME (PIXELS) | | |
|---|---|---|---|
| Regular | | | Total |
| wrinkles | | Fine wrinkles | (regular + fine) |
| UVB + 70% ethanol | 892,707 | 281,002 | 1,173,709 |
| UVB + 1% L-NAME | 896,863 | 175,295 | 1,072,158 |
| in 70% ethanol | | | |
| untreated control | 297,711 | 222,420 | 520,131 |

1. A method of preventing or treating a wrinkle in a subject, said method comprising administering to the subject a composition comprising an inhibitor of nitric oxide synthase (NOS) in an amount sufficient to reduce or prevent said wrinkle.

2. The method of claim 1, wherein the wrinkle is caused by exposure to UVB radiation.

3. The method of claim 1, wherein the wrinkle is a fine wrinkle.

4. The method of claim 1, wherein the inhibitor of NOS is administered topically.

5. The method of claim 1, wherein the inhibitor of NOS is provided in a sterile composition.

6. The method of claim 1, wherein the inhibitor of NOS is L-NAME.

7. A method of reducing a wrinkle in a subject, said method comprising identifying a subject in need of wrinkle reduction;
administering a NOS inhibitor compound to the subject; and
evaluating the effect of the administration on the wrinkle.

8. The method of claim 7, wherein the wrinkle is caused by exposure to UVB radiation.

9. The method of claim 7, wherein the wrinkle is a fine wrinkle.

10. The method of claim 7, wherein the NOS inhibitor is administered topically.

11. The method of claim 7, wherein the NOS inhibitor is provided in a sterile composition.

12. The method of claim 7, wherein the NOS inhibitor is L-NAME.

13. A composition for preventing or treating wrinkles comprising a NOS inhibitor and a pharmaceutically acceptable carrier.

14. The composition of claim 13, wherein the NOS inhibitor is L-NAME.

15. The composition of claim 13, wherein the weight percent of the L-NAME ranges from 0.01% to 10%.

16. The composition of claim 13, wherein the weight percent of the L-NAME ranges from 0.1% to 5%.

17. The composition of claim 13, further comprising a fragrance.

18. A method of providing wrinkle protection to a subject, said method comprising:
supplying to the subject a composition comprising a NOS inhibitor;
supplying to the subject instructions for using said composition to prevent or reduce wrinkles.

19. The method of claim 18, wherein the NOS inhibitor is L-NAME.

20. The method of claim 18, wherein the wrinkle protection is fine wrinkle protection.

21. The method of claim 18, wherein said instructions comprise directions to apply the composition to the skin prior to sun exposure.

22. The method of claim 18, wherein the composition further comprises a cosmetic ingredient.

23. A kit for preventing wrinkles in a subject, said kit comprising:
a composition comprising a NOS inhibitor; and
instructions for using the composition to prevent wrinkles.

24. The kit of claim 23, wherein said NOS inhibitor is L-NAME.

25. The kit of claim 23, wherein said wrinkles are fine wrinkles.

26. The kit of claim 23, wherein said composition further comprises a cosmetic ingredient.

27. The kit of claim 23, wherein said instructions comprise directions to apply said composition to the skin.
